(19) United States
(12) Patent Application Publication
(10) Pub. No.: US 2006/0036258 A1
(43) Pub. Date: Feb. 16, 2006

(54) Sizing distractor and method for implanting an interspinous implant between adjacent spinous processes

(75) Inventors: James F. Zucherman, San Francisco, CA (US); Ken Y. Hsu, San Francisco, CA (US); Charles A. Hartjen, Monkton, MD (US); Charles J. Winslow, Walnut Creek, CA (US); John Flynn, West Milford, NJ (US); David Bohrer, Barrington, RI (US)

Correspondence Address: FLIESLER MEYER, LLP, FOUR EMBARCADERO CENTER, SUITE 400, SAN FRANCISCO, CA, 94111, US

(73) Assignee: St. Francis Medical Technologies, Inc., Alameda, CA (US)

(21) Appl. No.: 11/147,816

(22) Filed: Jun. 8, 2005

Related U.S. Application Data
(60) Provisional application No. 60/578,026, filed on Jun. 8, 2004.

(51) Int. Cl. A61B 17/58

(52) U.S. Cl. 606/90

(57) ABSTRACT

Systems and methods in accordance with the present invention can be applied to distract adjacent interspinous processes and measure a distraction height, so that an appropriately sized implant can be positioned between interspinous processes to relieve pain associated with at least one of the spinal column and associated structures. A system in accordance with one embodiment can include a sizing distractor having distraction prongs extending from opposing jaws, the opposing jaws can taper laterally toward the distraction prongs to assist in positioning the distraction prongs between obstructive structures related to the spine.

### CLAIM TO PRIORITY

The present application claims the benefit of:

U.S. Provisional Patent Application No. 60/578,026 entitled SIZING DISTRACTOR AND METHOD FOR IMPLANTING AN INTERSPINOUS IMPLANT BETWEEN ADJACENT SPINOUS PROCESSES by Zucherman et al, filed on Jun. 8, 2004 (Attorney Docket KLYC-01053US3), the entire contents of which are incorporated herein by reference.

### CROSS-REFERENCE TO RELATED PATENTS AND PATENT APPLICATIONS

This U.S. Patent Application incorporates by reference all of the following:

U.S. patent application Ser. No. 09/999,754 entitled “INTERSPINOUS PROCESS IMPLANT SIZER AND DISTRACTOR WITH A SPLIT HEAD AND SIZE INDICATOR AND METHOD,” filed Oct. 31, 2001;

U.S. Provisional Patent Application No. 60/323,508 entitled “INTERSPINOUS PROCESS IMPLANT SIZER AND DISTRACTOR WITH A SPLIT HEAD AND SIZE INDICATOR AND METHOD,” filed Sep. 18, 2001;

U.S. patent application Ser. No. 09/799,470, entitled “SPINAL IMPLANTS, INSERTION INSTRUMENTS, AND METHODS OF USE,” filed Mar. 5, 2001;

U.S. patent application Ser. No. 09/799,215, entitled “SPINE DISTRACTION IMPLANT,” filed Mar. 5, 2001;

U.S. Pat. No. 6,235,030, entitled “SPINE DISTRACTION IMPLANT,” issued May 22, 2001 to Zucherman, et al.;

U.S. Pat. No. 6,190,387, entitled “SPINE DISTRACTION IMPLANT,” issued Feb. 20, 2001 to Zucherman et al.;
U.S. Pat. No. 6,068,630, entitled “SPINE DISTRACTION IMPLANT,” issued May 30, 2000 to Zucherman et al.; and
U.S. Pat. No. 6,048,342, entitled “SPINE DISTRACTION IMPLANT,” issued Apr. 11, 2000 to Zucherman, et al.

### TECHNICAL FIELD

The present invention relates to distraction of adjacent spinous processes and to sizing and/or implantation of an interspinous implant between adjacent spinous processes.

### BACKGROUND

As the present society ages, it is anticipated that there will be an increase in adverse spinal conditions which are characteristic of older people. By way of example only, with aging comes increases in spinal stenosis (including, but not limited to, central canal and lateral stenosis), the thickening of the bones which make up the spinal column and facet arthropathy. Spinal stenosis is characterized by a reduction in the available space for the passage of blood vessels and nerves. Pain associated with such stenosis can be relieved by medication and/or surgery. Additionally, pain associated with the spines of people of all ages can be caused by other ailments. Of course, in relieving spine related pain no matter what the cause, it is desirable to eliminate the need for major surgery for all individuals and in particular for the elderly.

Thus, there needs to be developed procedures, procedure instrumentations, and interspinous process implants for alleviating pain associated with the spine, which procedures and interspinous process implants are minimally invasive, can be tolerated by all patients and particularly by the elderly, and can be performed preferably on an outpatient basis.

### BRIEF DESCRIPTION OF THE DRAWINGS

FIG. 1A is a plan view of a sizing distractor in accordance with one embodiment of the present invention.

FIG. 1B is a close-up view of a sizing distractor in accordance with an alternative embodiment having needle-nose shaped jaws.

FIG. 1C is a close-up view of a sizing distractor in accordance with still another embodiment having concave shaped jaws.

FIG. 2A is a side view of the sizing distractor of FIG. 1A.

FIG. 2B is a side view of the distraction prongs of the sizing distractor positioned beside an interspinous implant.

FIG. 2C is a side view of the distraction prongs of a sizing distractor in accordance with an alternative embodiment of the present invention having a concave transition region.

FIG. 2D is a side view of the distraction prongs of a sizing distractor in accordance with still another embodiment of the present invention having a convex transition region.

FIG. 3A is a partial cross-sectional posterior view of the sizing distractor of FIG. 1A positioned between adjacent spinous processes.

FIG. 3B is a partial cross-sectional crania aspect view of the sizing distractor of FIG. 1A positioned between adjacent spinous processes.

FIG. 3C is a partial cross-sectional crania aspect view of a sizing distractor without a transition region positioned between adjacent spinous processes.

FIG. 4A is a side view of one embodiment of an interspinous implant for use in systems and methods in accordance with the present invention.

FIG. 4B is a side view of an alternative embodiment of an interspinous implant for use in systems and methods in accordance with the present invention.

FIG. 5 is a partial cross-sectional crania aspect view of a sizing distractor partially positioned between adjacent spinous process.

FIG. 6A is a partial cross-sectional side view of the sizing distractor of FIG. 1A partially inserted between adjacent spinous processes.

FIG. 6B is a partial cross-sectional side view of FIG. 6A with the sizing distractor fully inserted between adjacent spinous processes.

FIG. 6C is a partial cross-sectional side view of FIG. 6B with the sizing distractor fully inserted and distracted between adjacent spinous processes;

FIG. 6D is a partial cross-sectional side view of FIG. 6C with the sizing distractor just removed from between the spinous processes such that the interspinous ligament remains at least partially distracted.

FIG. 6E is a partial cross-sectional side view of FIG. 6D with an embodiment of an implant in accordance with the present invention inserted within the at least partially distracted spinous processes.

FIG. 7 is a partial cross-sectional posterior view of the implant of FIG. 6E positioned between spinous processes.

### DETAILED DESCRIPTION

Systems and methods in accordance with the present invention can be applied to distract adjacent interspinous processes, such that a customized implant can be implanted between the adjacent interspinous processes in order to relieve pain and discomfort in tissues and structures associated with the spine. The customized implant can limit extension of the spine in the implanted region, while freely allowing flexure of the spine.

Sizing Distractor

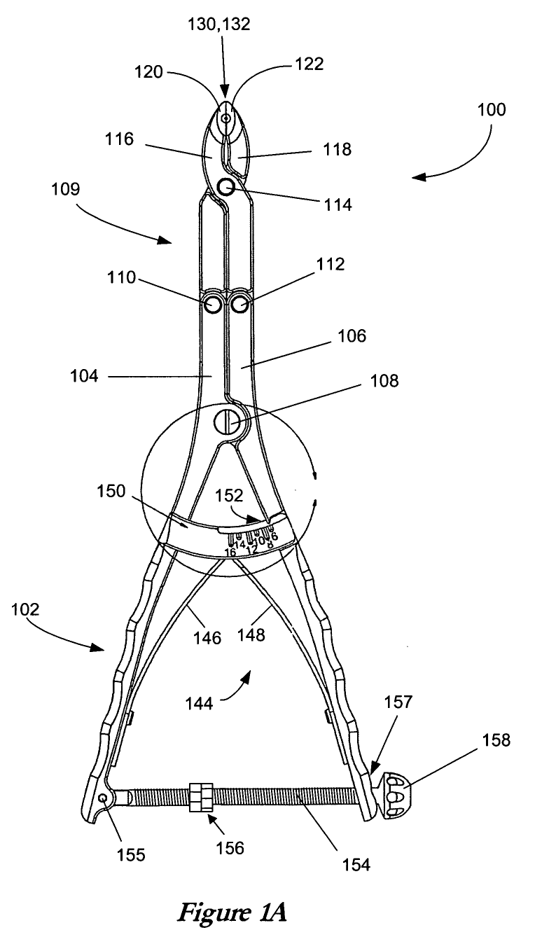

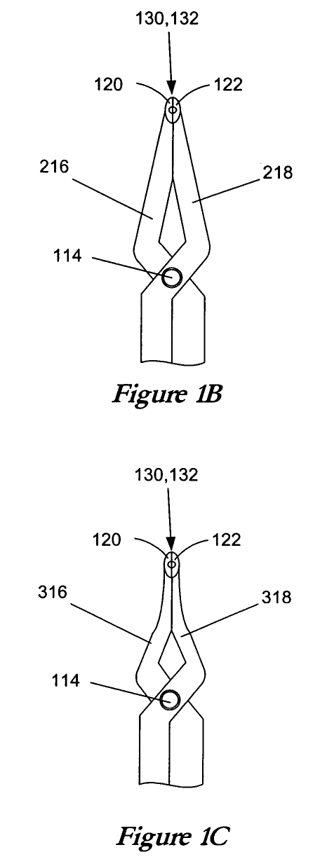

FIG. 1A is a plan view of an embodiment of a sizing distractor 100 in accordance with the present invention for use with embodiments of systems and methods of the present invention. Referencing for the figures is based on the point of insertion of the sizing distractor 100 between spinous processes so that at a distal end the sizing distractor 100 includes a first or lower distraction prong 120 and a second or upper distraction prong 122, and at a proximal end the sizing distractor 100 includes a handle (also referred to herein as a grip) 102. The handle 102 is defined by a first member 104 and a second member 106 pivotally connected by a fastener 108. As shown, the handle 102 is scalloped, or otherwise textured so that a physician can grip the handle 102 with less slippage than might otherwise occur. In other embodiments, the handle 102 need not be textured or scalloped, or can include some other feature for assisting proper handling. For example, in some embodiments, the handle 102 can include finger loops.

A leaf spring device 144 is connected with the first member 104 and the second member 106, and comprises a first single-leaf spring 146 and a second single-leaf spring 148 positioned in opposition to one another so that the leaf spring device 144 biases the handle 102 into an open position. The first and second single-leaf springs 146,148 are interlocked by positioning one of the first and second single-leaf springs 146,148 within a slot of the other of the first and second single-leaf springs 146,148. The distraction of the handle 102 is limited by the abutment of the first and second members 104,106 on the distal side of the pivot fastener 108. To spread the first distraction prong 120 and the second distraction prong 122 apart, the handle 102 is urged to a closed position. The leaf spring device 144 provides resistance when a physician squeezes, or otherwise urges the handle 102 closed, helping to control distraction of the distraction prongs 120,122 and helping to prevent unintended over-distraction. In other embodiments, a coil spring can be positioned between the first and second members 104,106, rather than a leaf spring device 144. In still other embodiments, some other biasing device can be used. One of ordinary skill in the art can appreciate the myriad different mechanical devices that can be employed to bias the handle 102 to an open position and provide resistance to closing the handle 102.

The distal end of the first member 104 and the distal end of the second member 106 are each associated with a corresponding jaw 116,118, the jaws being positioned in opposition to one another. In a preferred application of the sizing distractor 100 of the present invention, the sizing distractor 100 can be used to distract adjacent spinous processes. Such applications may require that the tool be inserted into a restricted space. A linkage 109 can be provided between the first and second members 104,106 and respective jaws 116,118 to extend the reach of the jaws 116,118. The linkage 109 can comprise a first hinge point 110, a second hinge point 112 and a jaw fastener 114. The first hinge point 110 pivotally connects the second jaw 118 and the first member 104, and the second hinge point 112 pivotally connects the first jaw 116 and the second member 106. The first and second jaws 116,118 are further pivotally connected with one another in a scissor-like fashion by the jaw fastener 114, translating a distracting motion of the first and second members 104,106 to a distracting motion of the first and second jaws 116,118. The amount of distraction ultimately applied to the jaws 116,118 can depend, for example, on a distance from the pivot fastener 108 to the hinge points 110,112, and the distance from the hinge points 110,112 to the jaw fastener 114. For example, if a finer distraction resolution is desired, the distance between the pivot fastener 108 and the hinge points 110,112 can be extended, or the distance from the hinge points 110,112 to the jaw fastener 114 can be reduced.

The first jaw 116 and the second jaw 118 each include grasping surfaces 130,132 that oppose and abut one another when the handle 102 is biased fully open. When the handle 102 is squeezed or otherwise urged closed, the first and second jaws 116,118 pivot apart. Each jaw 116,118 can have an arced shape from the jaw fastener 114 to the grasping surfaces 130,132 along the plane of distracting motion (i.e., the plane of the page of FIG. 1A) to provide a smooth shape with few edges or sharp lines for ease of insertion into an incision. However, the jaws 116,118 need not have an arced shape along the plane of distracting motion. In other embodiments, the jaws 116,118 can have some other shape. For example, as shown in FIG. 1B, in some embodiments the jaws 216,218 can have a needle-nose shape. In still further embodiments, the jaws 316,318 can have a concave shape, as shown in FIG. 1C. In still further embodiments, one jaw can have a shape different from the other jaw. One of ordinary skill in the art can appreciate the myriad different shapes with which the jaws can be formed.

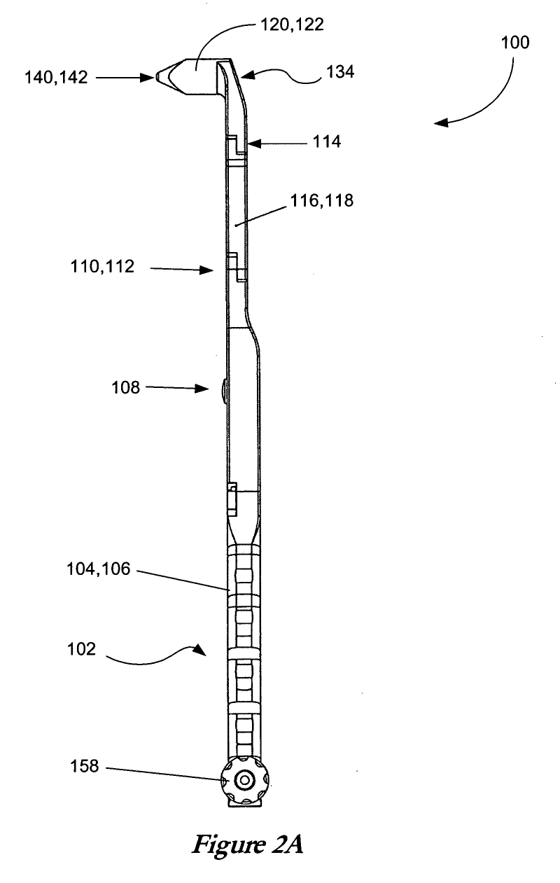

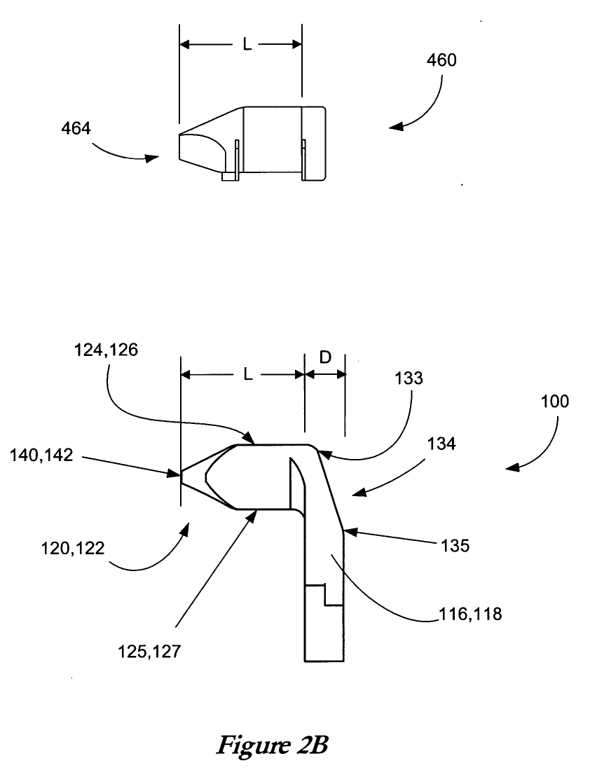

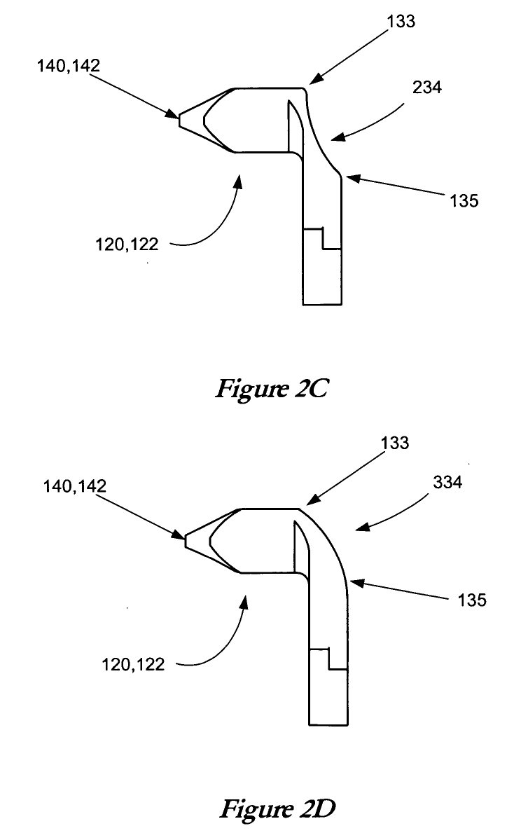

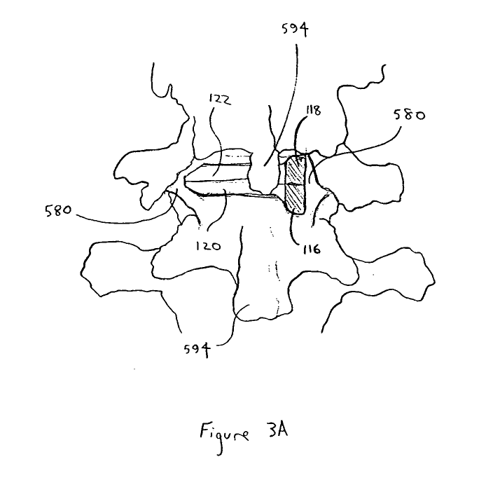

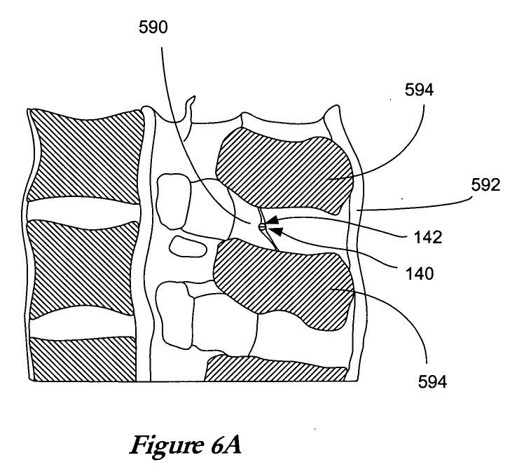

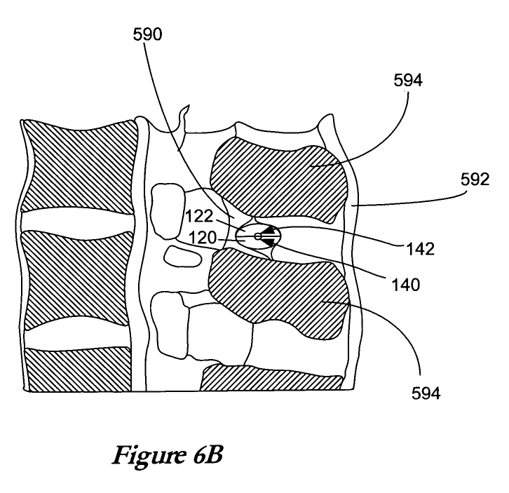

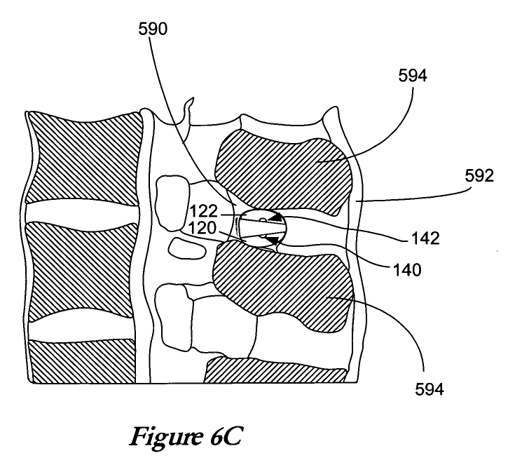

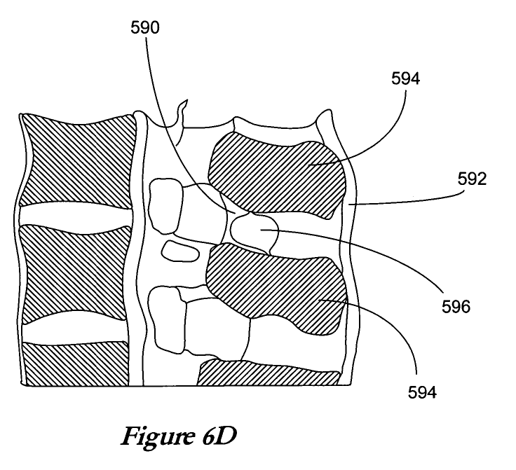

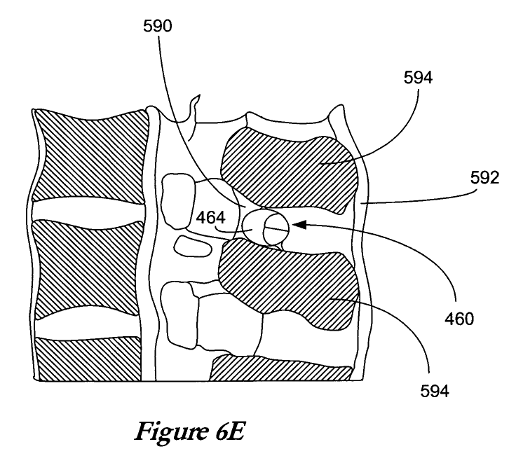

FIG. 2A is a right side view of the sizing distractor 100. The distraction prongs 120,122 extend laterally (along the axis of insertion) from the first and second jaws 116,118, respectively. The distraction prongs 120,122 roughly mirror one another and include surfaces extending from the grasping surfaces 130,132 of the first and second jaws 116,118 that similarly abut one another when the handle 102 is biased fully open. Referring to FIG. 2B, the distraction prongs 120,122 are sized substantially the same and have a length L that approximates the length of a body of an interspinous implant 460. Each distraction prong 120,122 has a distal end 140,142 adapted to be inserted between adjacent spinous processes. The distal ends 140,142 together define a tissue expander which is similar in design and formation to a tissue expander 464 of the implant 460. Thus, the distal ends 140,142 are tapered so that a portion of the distraction prong 120,122 having a relatively small cross-sectional area can initially pierce between or otherwise displace a taut interspinous ligament and can gradually further displace the interspinous ligament around the distraction prongs 120,122 as the distraction prongs 120,122 are further inserted, as shown in FIG. 6A-6E and described below.

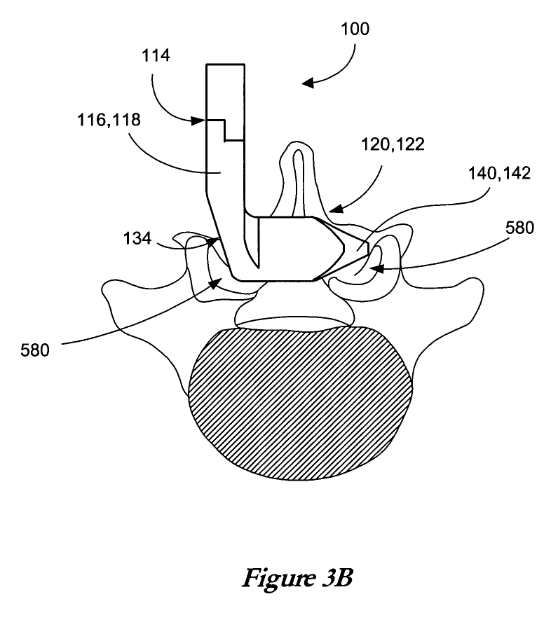

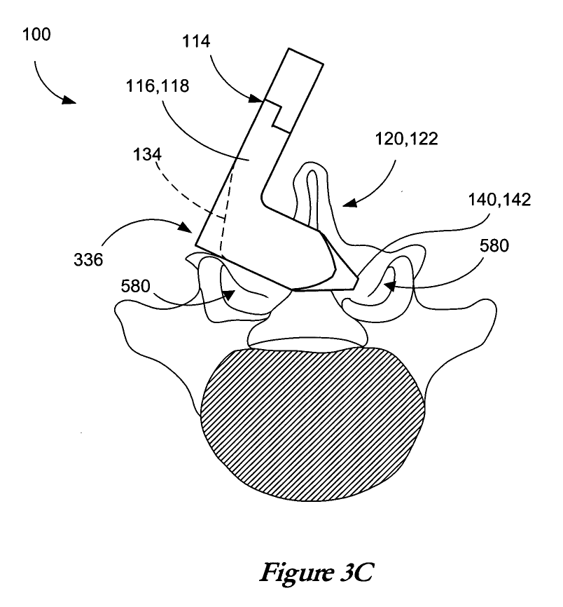

As can further be seen in FIG. 2B, the first and second jaws 116,118 include a transition region 134 that tapers laterally toward the distal ends 140,142 of the distraction prongs 120,122 so that an anterior edge 124,126 of the distraction prongs 120,122 approximates the implant length L. The transition region 134 can extend from a first location 135 along the jaws 116,118 proximally located relative to a posterior edge 125,127 of the distraction prongs 120,122 to a second location 133 located so that the anterior edge 124,126 approximates the implant length L. In other embodiments, the anterior edge 124,126 can be slightly longer or slightly shorter in length. Thus, when the distraction prongs 120,122 are positioned within an incision, the confined space located near the anterior portion of the spinous process need not accommodate both the length L of the distraction prongs 120,122 and the thickness D of the sizing distractor 100. The transition region 134 can improve clearance of the distraction prongs 120,122 between, for example, corresponding superior or inferior articular facets where positioned at the third through fifth lumbar vertebra, and/or corresponding mammillary processes where positioned at the first and second lumbar vertebra. Similar shapes would be appropriate at other locations in the spine relative to other spinous processes. For example, FIGS. 3A and 3B illustrate distraction prongs 120,122 positioned between spinous processes of the fourth and fifth lumbar vertebra. The tapered shape of the transition region 134 of the jaws 116,118 allows the distraction prongs 120,122 to be inserted between the adjacent spinous processes with minimum or no interference from the surrounding structures. In some embodiments, the transition region 134 can include a gradual and continuous taper. As can be seen in FIG. 2C, in other embodiments the transition region 234 can include a concave arc that provides increased clearance by more gradually increasing the width of the sizing distractor 100 from the first location 135 to the second location 133. Referring to FIG. 2D, in still other embodiments the transition region 334 can be curved, for example in a convex arc, to mimic a shape of the facets 580 (shown in FIG. 3B) between which the distraction prongs 120,122 are positioned. One of ordinary skill in the art can appreciate the many different shapes in which the transition region 134 can be formed

As can be seen in FIG. 3C, the structures associated with the spinous processes (e.g., the superior and/or inferior facets) can physically impede a physician's ability to position the distraction prongs 120,122 of the sizing distractor 100 between spinous processes where a transition region 134 is not associated with the jaws 114,116 of the sizing distractor 100. As shown, the heel 336 of the sizing distractor 100 contacts a superior articular facet 580, requiring the physician to, for example, raise the distal end 140,142 of the distraction prongs 120,122, which may cause undesired stretching of the interspinous ligament 590, and possibly the supraspinal ligament 592 (both shown in FIG. 6A-6E). This can make a surgical procedure more difficult and time-consuming. Increasing a time required to complete a surgical procedure can increase trauma to the patient. Further, without a transition region 134, the sizing distractor 100 may be more likely to cause damage to the spinous processes and surrounding structures through unintended or unavoidable contact.

In an embodiment in accordance with the present invention, the sizing distractor 100 can include a distraction gauge 150 connected with one of the first and second members 104,106. As shown in FIG. 1A, the distraction gauge 150 can be curved to correspond to an arc traced by the motion of the first and second members 104,106 pivoting about the pivot fastener 108. The other of the first and second members 104,106 can include a flag 152 or pointer for indicating a distraction height measurement on the distraction gauge 150. As the handle 102 is urged closed, the distraction gauge 150 slides past the flag 152, along with indicia indicating increasing distraction height. The distraction gauge 150 can indicate total distraction height of the spinous processes, and therefore can be calibrated to include a height of the first and second distraction prongs 120,122. For example, if the height of the first and second distraction prongs 120,122 combined is 6 mm, where the handle 102 is fully open (FIG. 1A) and the distraction prongs 120,122 abut one another, the flag 152 can indicate 6 mm, whereas when the handle 102 is urged closed and the distraction prongs 120,122 are distracted 8 mm, the flag 152 can indicate the total distraction of 14 mm. Alternatively, the distraction gauge 150 can indicate some other measure, for example a distance of distraction between the grasping surfaces 130,132. The measurement can be used to select an appropriately sized interspinous implant 460. For example, where the distraction gauge 150 measures 14 mm, an interspinous process implant 460 sized 14 mm can be selected for implantation.

As further shown in FIG. 1A, in one embodiment the sizing distractor 100 can include a threaded rod 154 connected at a pivot point 155 of one of the first and second members 104,106 and freely passing through a through-hole 157 in the other of the first and second members 104,106. As the handle 102 is urged closed, the threaded rod 154 passes through the through-hole 157 and pivots to follow the arcing travel of the handle 102. A distraction stop 156 can be positioned along the threaded rod 154 and sized such that the distraction stop 156 blocks the free travel of the threaded rod 154, preventing further closure of the handle 102 and limiting the maximum distraction height. As shown in FIG. 1A, the distraction stop 156 is fixed in position along the threaded rod 154, however, in other embodiments the distraction stop 156 can be adjustably positionable along the threaded rod 154 to allow the maximum distraction height to be adjusted. In still other embodiments, the threaded rod 154 need not include a distraction stop 156. A nut 158 can be associated with the threaded rod 154 at the free end of the threaded rod 154 such that when the nut 158 is advanced along the threads toward the pivot point 155, the nut 158 contacts the handle 102, fixing the handle 102 in position against the bias of the leaf spring device 144. Thus, a physician can urge the handle 102 closed to distract the distraction prongs 120,122 to a desired distraction height and fix the nut 158 against the handle 102 to maintain the distraction height. For small incremental changes, the nut 158 can be further twisted to urge the handle 102 closed or allow the handle 102 to open, depending on the direction of twist. In this way the nut 158 can permit careful and precise distraction of the distraction prongs 120,122. The pitch of the threads on the threaded rod 154 can be sized such that a desired level of precision can be obtained when the nut 158 is advanced along the threaded rod 154.

In other embodiments in accordance with the present invention, the distraction gauge 150 and threaded rod 154 can be a single device. For example, the threaded rod 154 can include indicia along the length of the threaded rod 154. One of the nut 158 and the one of the first and second members 104,106 through which the threaded rod 154 passes can include a flag or indicator. The indicia on the threaded rod 154 can be calibrated such that when the handle 102 is urged closed and the distraction prongs 120,122 are distracted, the distraction height can be measured. In still other embodiments, the sizing distractor 100 can include a pawl and ratchet mechanism in substitution of one or both of the threaded rod 154 and the distraction gauge 150. Sizing distractors 100 in accordance with the present invention can include myriad different devices for measuring distraction and/or fixing a range of distraction. The present invention is not intended to be limited in scope to the illustrations provided herein.

Sizing distractors 100 for use in systems and methods in accordance with the present invention can be made from medical grade metals. For example, the sizing distractor can comprise titanium or stainless steel. Thus, the sizing distractor 100 can be made sufficiently strong to support the force required to be applied during distraction of the spinous processes.

Interspinous Implant

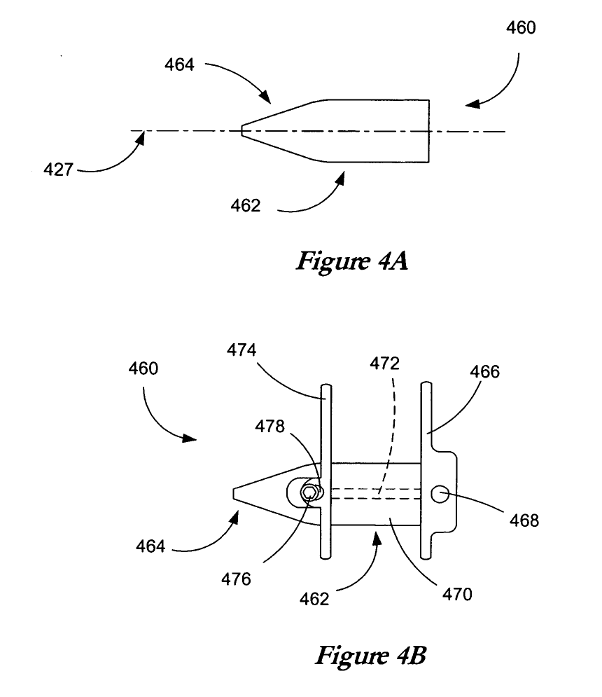

Systems and methods in accordance with the present invention can further comprise an interspinous implant positionable between spinous processes. Embodiments of interspinous implants for use in such systems and methods can include implants described in U.S. Pat. No. 6,068,630, issued May 30, 2000 to Zucherman, et al, incorporated herein by reference. For example, as shown in FIG. 4A, in one embodiment the interspinous implant 460 can include a distracting body 462 having a lead-in tissue expander 464 connected with or extending from a distal end of the distracting body 462. The lead-in tissue expander 464 acts as a distraction guide to initiate distraction of the soft tissue and adjacent spinous processes when the implant 460 is surgically inserted between the spinous processes. As described above, a length of the distracting body 462 and lead-in tissue expander 464 can be approximately the same as the length L of the distraction prongs 120,122 of the sizing distractor 100. The lead-in tissue expander 464 can have a tapered shape to ease insertion of the implant 460 between the spinous processes. As shown, the lead-in tissue expander 464 tapers substantially along the longitudinal axis 427, although in other embodiments the lead-in tissue expander 464 can have some other shape. For example, in one embodiment the lead-in tissue expander 464 can be shaped more similarly to the distal ends of the closed distraction prongs 120,122, as shown in FIG. 3B.

As shown in FIG. 4B, in other embodiments the interspinous implant 460 can include a wing 466, from which the distracting body 462 extends. The wing 466 can assist in positioning the interspinous implant 460 between spinous processes, and can limit lateral movement of the implant 460 once the implant 460 is in position. The wing 466 can include slots or holes 468, so that an implant insertion tool can grasp the interspinous implant 460. The distracting body 462 can further comprise a shaft 472 extending from the wing 466, upon which is mounted a rotatable spacer 470, the rotatable spacer 470 being rotatable relative to the shaft 472. The rotatable spacer 470 can have an elliptical cross-section having a major dimension and a minor dimension, the minor dimension corresponding to the desired distraction height of the spinous processes. As can be seen in the cross-sections of FIG. 6A-6F, the spinous processes 594 can include upper and lower surfaces that are not flat, but that include some surface features. The rotatable spacer 470 is free to rotate relative to the wing 466, allowing the interspinous implant 460 to settle into an appropriate orientation, in order to accommodate the space in which it is positioned. In other embodiments, the rotatable spacer 470 can have a cross-sectional shape other than elliptical. For example, the rotatable spacer 470 can have a teardrop, wedge, circular, oval, ovoid, football, or other cross-sectional shape.

The interspinous implant 460 can further include a second wing 474 adjustably connectable with the lead-in tissue expander 464 by a fastener 476 such that the adjacent spinous processes can be positioned between the first wing 466 and the second wing 474. Where the interspinous implant 460 comprises a second wing 474, the lead-in tissue expander 464 can include a slot 478 having a threaded hole, the slot 478 allowing sliding movement of the second wing 474 along the longitudinal axis 427 relative to the first wing 466. In one embodiment, the second wing 474 can have a shape substantially similar to that of the first wing 466, while in other embodiments, the second wing 474 can have a different shape than that of the first wing 466.

The interspinous process implant 460 can be made from a variety of medical grade stainless steel alloys and cobalt chrome, both of which are well known materials as candidates for medical implants that are load-bearing. Additionally, another material considered to rank highly across a number of desirable attributes such as strength, and biocompatibility is medical grade titanium, and alloys thereof.

In addition to metals, it is further contemplated that interspinous process implant 460 can be made in whole or in part from somewhat flexible and/or deflectable material. In these embodiments, the implant and/or portions thereof can made out of a polymer, more specifically, the polymer can be a thermoplastic. Still more specifically, the polymer can be a polyketone known as polyetheretherketone (PEEK). Still more specifically, the material can be PEEK 450G, which is an unfilled PEEK approved for medical implantation available from Victrex of Lancashire, Great Britain. Other sources of this material include Gharda located in Panoli, India. The implant and/or portions thereof can be formed by extrusion, injection, compression molding and/or machining techniques. The material specified has appropriate physical and mechanical properties and is suitable for carrying and spreading the physical load between the spinous process. Further in this embodiment, the PEEK has the following additional approximate properties:

| Property | Value | |
|---|---|---|
| Density | 1.3 | g/cc |
| Rockwell M | 99 | |
| Rockwell R | 126 | |
| Tensile Strength | 97 | MPa |
| Modulus of Elasticity | 3.5 | GPa |
| Flexural Modulus | 4.1 | GPa |

In some embodiments, the implant can be comprised, at least in part, of titanium or stainless steel, or other suitable implant material which is radiopaque and, at least in part, of a radiolucent material that does not show up under x-ray or other type of imaging. For example, in one embodiment, the first wing 466 and second wing 474 and the shaft 472 are comprised of a radiopaque material (e.g., titanium) and the rotatable spacer 470 and lead-in tissue expander 464 are comprised of a radiolucent material (e.g., PEEK). In such an embodiment, under imaging the implant looks like an “H”. The physician can have a less obstructed view of the spine under imaging, than with an implant comprised entirely of radiopaque materials. However, the implant 460 need not comprise any radiolucent materials.

It should be noted that the material selected can also be filled. For example, other grades of PEEK are available and contemplated, such as 30% glass-filled or 30% carbon-filled, provided such materials are cleared for use in implantable devices by the FDA, or other regulatory body. Glass-filled PEEK reduces the expansion rate and increases the flexural modulus of PEEK relative to that which is unfilled. The resulting product is known to be ideal for improved strength, stiffness, or stability. Carbon-filled PEEK is known to enhance the compressive strength and stiffness of PEEK and lower its expansion rate. Carbon-filled PEEK offers wear resistance and load carrying capability.

As will be appreciated, other suitable similarly biocompatible thermoplastic or thermoplastic polycondensate materials that resist fatigue, have good memory, are flexible, and/or deflectable, have very low moisture absorption, and good wear and/or abrasion resistance, can be used without departing from the scope of the invention. For example, in one embodiment the spacer 370 can comprise polyetherketoneketone (PEKK). Other material that can be used include polyetherketone (PEK), polyetherketoneetherketoneketone (PEKEKK), and polyetheretherketoneketone (PEEKK), and generally a polyaryletheretherketone. Further, other polyketones can be used as well as other thermoplastics.

Reference to appropriate polymers that can be used in the implant can be made to the following documents, all of which are incorporated herein by reference. These documents include: PCT Publication WO 02/02158 A1, dated Jan. 10, 2002, entitled “Bio-Compatible Polymeric Materials;” PCT Publication WO 02/00275 A1, dated Jan. 3, 2002, entitled “Bio-Compatible Polymeric Materials;” and, PCT Publication WO 02/00270 A1, dated Jan. 3, 2002, entitled “Bio-Compatible Polymeric Materials.”

In still other embodiments, materials such as Bionate®, polycarbonate urethane, available from the Polymer Technology Group, Berkeley, Calif., may also be appropriate because of the good oxidative stability, biocompatibility, mechanical strength and abrasion resistance.

Operation of Sizing Distractor

FIG. 6A is a lateral view in partial cross-section of a portion of the vertebral column comprising adjacent spinous processes 594. Interspinal ligaments 590 interconnect adjacent spinous processes 594 from the root to the apex of each spinous process 594. The interspinal ligaments 590 are thin and membranous, and can be displaced with the distraction prongs 120,122 of the sizing distractor 100. As described above, the sizing distractor 100 is used to distract the spinous processes 594 and to facilitate insertion of an interspinous implant 460 between the spinous processes 594. In order to accomplish distraction and implantation, a suitable incision can be made and sized to access a targeted region of the spine. The sizing distractor 100 can be inserted into the incision and positioned along the interspinous ligament 590 such that the distal ends 140,142 of the distraction prongs 120,122 are approximately aligned with a target location for the interspinous implant 460. The distal ends 140,142 of the distraction prongs 120,122 are then urged between the spinous processes 594, such that the distraction prongs 120,122 pierce or otherwise displace the interspinous ligament 590. As can be seen in FIG. 6B, as the distraction prongs 120,122 are further urged between the spinous processes 594, the fibrous interspinous ligament 590 is displaced around the distraction prongs 120,122.

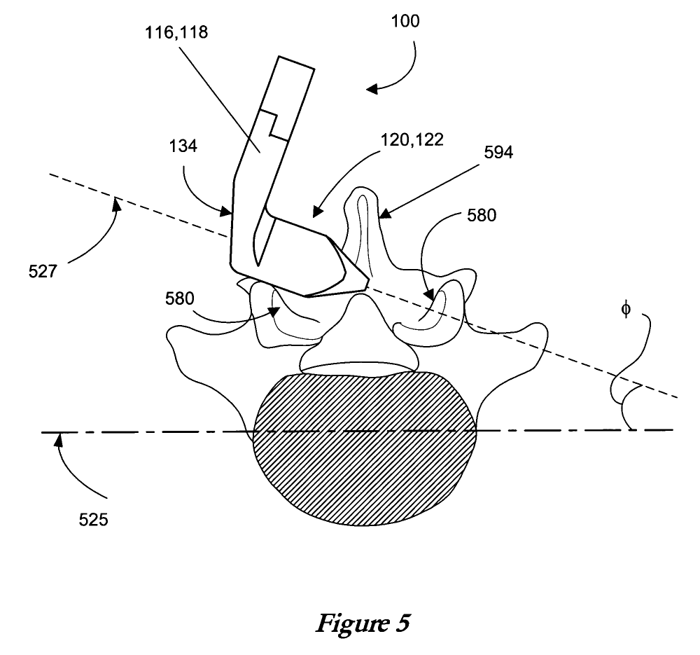

Referring to FIG. 5, the distraction prongs 120,122 can be initially urged between the spinous processes 594 so that the longitudinal axis 527 of the distraction prongs 120,122 forms an angle □ relative to the coronal plane 525 to avoid contacting the superior articular facets 580 on either side of the interspinous ligament (not shown in FIG. 5). (The arrangement of spinous processes relative to related structures can vary along the spinal column. For example, where the sizing distractor 100 is positioned between spinous processes connected with different vertebrae than those illustrated, the sizing distractor 100 will be positioned between structures other than superior facets 580. Thus, in other embodiments of methods and sizing distractors 100 in accordance with the present invention, the sizing distractor 100 can be positioned to avoid protruding or potentially obstructing structures such as inferior articular facets and/or mammillary processes.) As the distraction prongs 120,122 are further urged between the spinous processes 594 and through the interspinous ligament 590, the transition region 134 of the jaws 116,118 of the sizing distractor 100 physically clears the superior facet 580 (or other structure), and the distraction prongs 120,122 can align along the coronal plane 525 (as shown in FIG. 3B).

Once the distraction prongs 120,122 are positioned, the physician can squeeze or otherwise urge the handle 102 closed, causing the distraction prongs 120,122 to separate and distract, as shown in FIG. 6C. The distraction prongs 120,122 can be distracted to a height such that the distraction prongs 120,122 contact the adjacent spinous processes 594, or distract the adjacent spinous processes 594, as desired. The distraction height can be measured to determine an appropriate size of interspinous implant 460 to be implanted, for example using the distraction gauge 150. The distraction prongs 120,122 are held in a distracted position until a physician determines that the interspinous ligament 590 will retain at least a portion of its displaced shape for a desired time sufficient to allow implantation of the interspinous implant 460. The distraction prongs 120,122 can be held in position, optionally, by setting the nut 158 so that the nut 158 abuts the handle 102 along the threaded rod 154, thereby opposing the bias of the leaf-spring device 144 and a force created by the resistance of the distracted spinous processes 594 to distraction. Once the desired time has lapsed, the handle 102 can be released, or otherwise allowed to return to a biased-open position, with the distraction prongs 120,122 closed. The distraction prongs 120,122 can then be removed from between the spinous processes 594, and the sizing distractor 100 can be removed from the incision. The spinous ligament 590 temporarily remains at least partially displaced, as shown in FIG. 6D, and relaxes over a period of time, allowing the physician to position an implant 460 within the interspinous ligament.

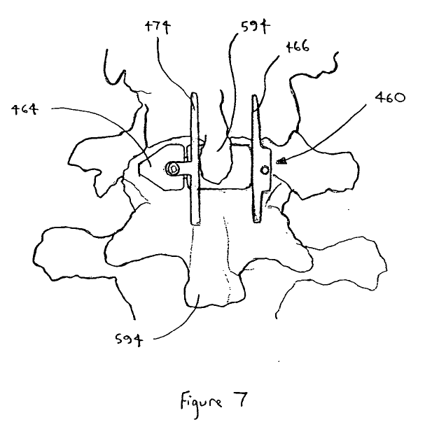

Once the sizing distractor 100 is removed from the incision, an appropriate interspinous implant 460 can be positioned between the spinous processes 594 within the opening 596 created by the displacement of the interspinous ligament 590. To position the interspinous implant 460, the first wing 466 can be grasped by a first insertion tool and inserted into the incision. The interspinous implant 460 is then positioned so that the lead-in tissue expander 464 can be inserted through the interspinous ligament 590. The interspinous implant 460 is then urged between the spinous processes 594 until appropriately positioned, as show in FIG. 6E. Once the interspinous implant 460 is properly positioned, a second insertion tool grasping a second wing 474 can then be optionally inserted into the incision, and the second wing 474 can be fastened to the lead-in tissue expander 464 so that the spinous processes 594 are sandwiched between the first and second wing 466,474, as seen in FIG. 7. The first and second insertion tools are used cooperatively to align and fasten the second wing 474 to the implant 460. The insertion tools can be removed from the incision, and the incision can be closed.

A sizing distractor 100 in accordance with embodiments of the present invention can be employed to distract adjacent spinous processes 594 to variable distraction heights, and can permit a physician to measure such distraction heights to determine an appropriate interspinous implant 460 for positioning between the corresponding spinous processes 594. The variable distractibility of the sizing distractor 100 can render unnecessary the use of a plurality of feeler gauges, or the like, for displacing the interspinous ligament 590 and determining an appropriate size of an interspinous implant 460 for implantation. For example, such a surgical procedure can commonly require distracting the spinous processes 594 with a plurality of feeler gauges. It is likely that a first feeler gauge can underestimate a desired distraction of targeted spinous processes 594, or that a first feeler gauge can simply initiate displacement of an interspinous ligament 590 between target adjacent spinous processes 594. Such a procedure can require the use of additional, progressively larger feeler gauges incrementally applied to achieve a desired distraction. A feeler gauge must be inserted and removed from the incision before replacing it with the next larger feeler gauge (or smaller feeler gauge, where over-distraction occurs). Such a procedure can be time consuming. Once the desired distraction is achieved, the final feeler gauge can be removed from the incision and a corresponding implant can be selected for implantation. Employing a single sizing distractor 100 in accordance with embodiments of the present invention in such surgical procedures can simplify and reduce the time required to complete the surgical procedure compared with the use of multiple tools.

The foregoing description of the present invention has been presented for purposes of illustration and description. It is not intended to be exhaustive or to limit the invention to the precise forms disclosed. Many modifications and variations will be apparent to practitioners skilled in this art. The embodiments were chosen and described in order to best explain the principles of the invention and its practical application, thereby enabling others skilled in the art to understand the invention for various embodiments and with various modifications as are suited to the particular use contemplated. It is intended that the scope of the invention be defined by the following claims and their equivalents.

What is claimed:

1. A sizing distractor adapted to distract spinous processes so that an implant can be positioned between the spinous processes for relieving pain associated with at least one of the spinal column and associated tissues and structures, the sizing distractor comprising:
a handle;
a jaw associated with said handle, a portion of the jaw having an initial thickness;
a distraction prong extending from said jaw, said distraction prong having an anterior edge, a posterior edge, and a distal end adapted to be inserted between the adjacent spinous processes; and
wherein said jaw includes a transition, the transition being a taper at least from the posterior edge to the anterior edge and having a thickness that varies from the initial thickness to a final thickness.

2. A system to relieve pain associated with at least one of the spinal column and associated tissues and structures, the system comprising:
a sizing distractor adapted to distract spinous processes, the sizing distractor including:
a handle;
a jaw associated with said handle, a portion of the jaw having an initial thickness;
a distraction prong extending from said jaw along an axis of insertion, said distraction prong having an anterior edge, a posterior edge, and a distal end adapted to be inserted between the adjacent spinous processes; and
wherein said jaw and said distraction prong have a combined length along the axis of insertion that varies from the posterior edge to the anterior edge.

3. The system of claim 2, wherein:
said jaw is a first jaw;
said distraction prong is a second distraction prong;
said anterior edge is a first anterior edge;
said posterior edge is a first posterior edge; and
said sizing distractor further includes:
a second jaw associated with said handle, a portion of the second jaw having the initial thickness;
a second distraction prong extending from said second jaw along the axis of insertion, said second distraction prong having a second anterior edge, a second posterior edge, and a second distal end adapted to be inserted between the adjacent spinous processes; and
wherein said second jaw and said second distraction prong have a combined length along the axis of insertion that varies from the posterior edge to the anterior edge.

4. The system of claim 3, wherein said handle is adapted to be manipulated such that said first distraction prong and said second distraction prong are urged apart.

5. The system of claim 3, wherein said sizing distractor further includes a distraction gauge.

6. The system of claim 3, wherein said handle further includes:
a threaded rod pivotally connected with said handle; and
a nut operably associated with said threaded rod;
wherein said nut is adapted to fix said first distraction prong and said second distraction prong in a distracted position.

7. The system of claim 3, wherein the handle includes a scissors mechanism that can selectively distract said first distraction prong and said second distraction prong.

8. The system of claim 7, wherein said handle further includes:
a threaded rod pivotally connected with said handle; and
a nut operably associated with said threaded rod;
wherein said nut is adapted to fix said first distraction prong and said second distracting in a distracted position.

9. The system of claim 3, wherein:
a portion of said first distal end of said first distraction prong is tapered; and
a portion of said second distal end of said second distraction prong is tapered.

10. The system of claim 3, wherein:
a first portion of a tissue expander is associated with said first distal end of said first distracting; and
a second portion of a tissue expander is associated with said second distal end of said second distraction prong.

11. The system of claim 2, further comprising:
an implant positionable between the adjacent spinous processes, the implant including:
a first wing with a body extending therefrom, said central body having a longitudinal axis;
said body including a sleeve, said sleeve being able to rotate about said longitudinal axis in order to aid in positioning said implant between spinous processes; and
a tissue expander associated with a distal end of said body, said body and tissue expander extending a length.

12. The system of claim 11, wherein said tissue expander is tapered.

13. The system of claim 11, wherein said sleeve has an elliptical cross-section in a plane which is substantially perpendicular to said longitudinal axis.

14. The system of claim 11, further comprising a second wing adapted to be connected with said implant such that said sleeve is positioned between said first wing and said second wing.

15. The system of claim 11 wherein the said distraction prong is substantially in the shape and size of said body and said tissue expander.

16. The sizing distractor of claim 2 wherein the distraction prong is of a shape and size that the distraction prong is adapted to fit between adjacent spinous processes and facets associated with the adjacent spinous processes.

17. A method for inserting an interspinous process implant between adjacent spinous processes and between a corresponding pair of facets, comprising:
selecting a sizing distractor having:
a first jaw,
a first distraction prong extending from the first jaw and having a first anterior edge and a first posterior edge,
wherein the first jaw tapers laterally toward said distraction prong such that the first anterior edge of said first distraction prong has a length substantially similar to the first posterior edge of said first distraction prong,
a second jaw,
a second distraction prong extending from the second jaw and having a second anterior edge and a second posterior edge, and
wherein the second jaw tapers laterally toward said second distraction prong such that the second anterior edge of said second distraction prong has a length substantially similar to the second posterior edge of said second distraction prong;
creating an access point to adjacent first and second spinous processes;
positioning said sizing distractor in said access point;
inserting said first distraction prong and said second distraction prong between said first and second spinous processes such that a portion of an interspinal ligament connected between said first and second spinous processes is displaced;
urging said first distraction prong and said second distraction prong apart thereby distracting said spinous processes to a desired distraction height;
choosing an interspinous process implant to correspond to the desired distraction height and that has a length that is a length of one of the first distraction prong and the second distraction prong;
removing said sizing distractor from between said first and second spinous processes; and
implanting the interspinous process implant between the spinous processes.

18. The method of claim 17, wherein said sizing distractor further includes a threaded rod pivotally connected with said handle; and a nut operably associated with said threaded rod;
the method includes the steps of:
using the nut to measure a distraction height.

19. The method of claim 18, further comprising:
adjusting said nut so that said nut fixes said first distraction prong and said second distraction prong in a distracted position.

20. The method of claim 18, further comprising:
adjusting said nut so that said nut urges said first distraction prong and said second distraction prong apart.
